(19) United States
(12) Patent Application Publication
(10) Pub. No.: US 2019/0143012 A1
(43) Pub. Date: May 16, 2019

(54) APPARATUS TO COLLECT AND MIX A HEMATIC FLUID

(71) Applicant: DELCON S.R.L., Milano (IT)

(72) Inventor: Norberto SALA, Arcore (IT)

(73) Assignee: DELCON S.R.L., Milano (IT)

(21) Appl. No.: 16/310,346

(22) PCT Filed: Jun. 16, 2017
(86) PCT No.: PCT/EP2017/064774

§ 371 Date: Dec. 14, 2018

(30) Foreign Application Priority Data
Jun. 16, 2016 (IT) 102016000062355

(51) Int. Cl. A61M 1/02

(52) U.S. Cl. CPC A61M 1/0245; A61M 2205/3569; A61M 2205/6054; A61M 2205/502; A61M 2205/3592

(57) ABSTRACT

Apparatus to collect and mix a hematic fluid, comprising a main body, on which at least one tray is positioned, able to receive and support at least a sac connected fluidically to a hematic fluid transfer tube, and at least a user interface to input and display data; the user interface is associated with the main body by means of removable connection means configured to allow the user interface to be disposed in a plurality of different positions with respect to the main body of the apparatus, so that the apparatus can assume different operating configurations.

### FIELD OF THE INVENTION

The present invention concerns an apparatus to collect and mix a determinate volume or weight of hematic fluid, usable for example in the medical field, in particular to collect and mix blood, hematic portions and/or hemo-derivatives, more particularly during procedures to transfer/collect said hematic fluid.

### BACKGROUND OF THE INVENTION

It is known that in the medical field of blood transfusion, the blood or hematic fractions (hemo-derivatives and/or hemo-components), or in general hematic fluids, can be taken from and/or respectively supplied to the patient using sacs, normally containing an anticoagulant, connected to plastic tubes inside which the blood or hematic fractions flow.

For example, in the case of blood donation, the donor is made to lie down on a chair-bed, a tourniquet is attached to one arm and a needle is inserted into a vein. The needle is connected to a tube, in turn connected to a blood collection sac. The blood spontaneously flows out until it fills the collection sac which already contains an anticoagulant solution or liquid and other substances or solutions useful for optimum storage of the blood. Before the needle is extracted at the end of the donation, some test tubes are filled up to carry out the tests provided by the law. The volume of blood withdrawn is generally established by law, to ensure proper preparation of the hemo-components (concentrates of red blood cells, platelets, plasma units), and also to ensure the absence of complications for the donor.

In this context, it is therefore necessary to measure the flow of hematic fluid that is removed and/or supplied from/to the patient, quantifying the volume of fluid present in the sacs. It is required that this quantification be sufficiently precise, reliable and repeatable over time.

It is also known that the sacs must be kept in continuous motion during filling with hematic fluid, in order to mix the hematic fluid during the transfer/collection procedures in the sacs, and to prevent it from coagulating.

Known collection and mixing apparatuses of a hematic fluid generally comprise a main casing or body with which a mobile tray is associated, on which the sacs to be weighed rest, and a user interface provided with data display and insertion elements.

A first disadvantage of known apparatuses is that the user interface, which can consist for example of a display and suitable data input keys, is indissolubly connected to the main casing or body of the apparatus.

This indissoluble connection of the user interface with the casing of the apparatus naturally causes obvious disadvantages from the point of view of the efficiency and ergonomics of the use of the apparatus. The apparatus, in fact, is generally positioned lower than the patient's arm, so as to generate a flow of hematic fluid by gravity and allow to fill the collection sac. The interface, in this case, finds itself in a position that is practically invisible to the user and not accessible to the operators.

Another disadvantage of known blood collection and mixing apparatuses is the poor efficiency and flexibility of use of the clamp, or closing gripper, of the tube where the hematic fluid passes. In particular, known clamps show defects in the correct positioning of the tube and in the absence of safety on the closing of the flow of hematic fluid at the end of the donation or in an emergency. The clamp is generally positioned on the main body of the apparatus in a position close to the tray.

Another disadvantage of known apparatuses, which again causes a poor and inefficient operating efficiency, is also connected to the lack of means that can allow an automatic recognition of a certain collection sac or of each of the collection sacs on the tray, and hence of the donor associated with them. The collection sacs are part of a collection kit.

Other limitations and disadvantages of conventional solutions and technologies will be clear to a person of skill after reading the remaining part of the present description with reference to the drawings and the description of the embodiments that follow, although it is clear that the description of the state of the art connected to the present description must not be considered an admission that what is described here is already known from the state of the prior art.

There is therefore a need to obtain an apparatus to collect and mix a hematic fluid that can overcome at least one of the disadvantages of the state of the art.

One purpose of the present invention is therefore to obtain an apparatus to collect and mix a hematic fluid that is extremely flexible and effective in order to function according to different operating types.

Another purpose of the present invention is to obtain an apparatus to collect and mix a hematic fluid which has great advantages in terms of practicality and ergonomics of use compared to the apparatuses of the state of the art.

Another purpose of the present invention is to obtain an apparatus to collect and mix a hematic fluid that is extremely effective and rapid in all the various operating steps of collecting the hematic fluid, mixing it and recognizing each of the sacs used for collection.

Another purpose of the present invention is to obtain an apparatus to collect and mix a hematic fluid that can guarantee an effective and safe management of the clamp or closing gripper of the tube through which the hematic fluid passes.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purposes, an apparatus to collect and mix a hematic fluid comprises a main body on which at least one tray is positioned, able to receive and support at least a sac connected fluidically to a hematic fluid transfer tube, and at least a user interface to input and display data. According to one aspect of the invention, the user interface is associated with the main body by means of removable connection means configured to allow the user interface to be disposed in a plurality of different positions with respect to the main body of the apparatus, so that the apparatus can assume different operating configurations.

According to another aspect of the invention, the user interface comprises a box-like body removably associable with a support unit integrated with the main body.

In some embodiments, the main body comprises one or more support rods configured to house the user interface in a raised position.

The present apparatus can comprise a support able to be positioned on a board or slider provided with a first plane where the main body can be positioned and a second plane where the user interface can be positioned, or vice versa.

The main body can be provided with seatings for the insertion of an appendix integrated with the user interface.

The apparatus comprises a clamp that can be configured to cooperate with the hematic fluid transfer tube and can be provided with at least a mobile division disposed inside a positioning slit of the tube and associated with corresponding drive means; the mobile division is configured to assume at least a first inactive position, at least an intermediate holding position of the tube, in which a flow of hematic fluid is allowed, and at least a clamping position of the tube, to interrupt the flow of hematic fluid.

In some embodiments, the clamp is positioned on the support unit integrated with the main body of the apparatus.

According to another aspect of the invention, the apparatus comprises at least an RFID communication module installable in proximity to the tray and able to read corresponding electronic labels which can be positioned on the hematic fluid collection sacs, in order to carry out an automatic recognition of each sac that transits on the tray.

In some embodiments, the communication module comprises an RFID antenna that is disposed in proximity to the tray and preferably in proximity to every side thereof, so as to substantially cover the entire area and allow the reading of one or more collection sacs.

The tray can advantageously comprise a magnetic device for connection to the main body.

These and other aspects, characteristics and advantages of the present disclosure will be better understood with reference to the following description, drawings and attached claims. The drawings, which are integrated and form part of the present description, show some forms of embodiment of the present invention, and together with the description, are intended to describe the principles of the disclosure.

The various aspects and characteristics described in the present description can be applied individually where possible. These individual aspects, for example aspects and characteristics described in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWINGS

These and other characteristics of the present invention will become apparent from the following description of some embodiments, given as a non-restrictive example with reference to the attached drawings wherein:

FIG. 1 is a three-dimensional view of an apparatus to collect and mix a hematic fluid according to the invention with a removable user interface;

FIG. 2 is a three-dimensional view of a first positioning mode of the user interface in the present apparatus;

FIG. 3 is a three-dimensional view of a second positioning mode of the user interface in the present apparatus;

FIG. 4 is a three-dimensional view of a third positioning mode of the user interface in the present apparatus;

FIGS. 5a and 5b are three-dimensional views of a first executive variant of the present invention;

FIG. 6 is a three-dimensional view of another executive variant of the present invention;

FIG. 7 shows a detail of the executive variant of the apparatus of FIG. 6 on a larger scale;

FIGS. 8a, 8b, 8c show a clamp or closing gripper of the passage tube of a hematic fluid in three operating positions;

FIG. 9 shows a three-dimensional view of the present apparatus provided with a system to read and automatically recognize the hematic fluid sacs.

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We shall now refer in detail to the various embodiments of the present invention, of which one or more examples are shown in the attached drawings. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

Embodiments described here concern an apparatus 10 to collect and mix hematic fluid that can be used in any possible transfer/collection procedure of a desired amount, weight or volume of hematic fluid in a collection sac, not shown.

The apparatus 10 comprises a tray 11, able to receive and support a collection sac fluidically connected to a hematic fluid transfer tube.

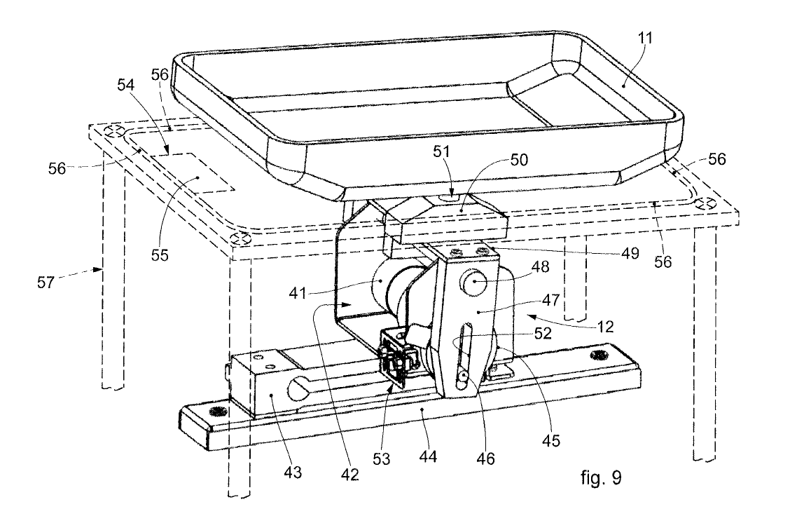

The tray 11 is connected to a movement unit 12, see FIG. 9, housed inside a main body 13, which substantially represents the casing of the apparatus.

As will be seen, the movement unit 12 has the function of moving the tray 11 so as to determine the mixing of the hematic fluid in the collection sac.

By way of example, the movement unit 12 is of the type able to allow a two-dimensional oscillation of the tray 11, but could also be of a different type.

FIG. 9 shows a type of movement unit 12 able to allow two-dimensional oscillation of the tray 11, however, the movement unit 12 could be made differently, provided it always guarantees at least said two-dimensional oscillation.

The tray 11 is positioned on a seating 14, made outside and above the main body 13.

The seating 14 will comprise an aperture for connecting the tray 11 to the movement unit 12 contained inside the main body 13.

The main body 13 also houses a sensor unit 43 inside it (FIG. 9), with the purpose of detecting the weight force acting on the tray 11, that is, detecting the weight and therefore also the volume of hematic fluid contained in the collection sac.

Another seating 15 is made on the main body 13, on which a user interface 16 at least partly rests.

The user interface 16 comprises a box-like body 17 on which a monitor 18 is made, for example a touchscreen monitor 18, by means of which it is possible to insert and display data relating to the collection of hematic fluid and in general relating to the functioning of the apparatus 10.

As an alternative to the touchscreen monitor 18, a user interface 16 can be provided with separate data display and input elements such as a display and a series of keys.

The user interface 16 will accommodate a control board inside it.

The user interface 16 can also be provided with a USB port 19 or suchlike, for connection to other devices, for example data storage devices, data processors, or suchlike.

The USB port 19, or suchlike, can also be disposed in any other position of the apparatus 10, for example on the main body 13. Also, a plurality of USB ports 19 or suchlike could be provided.

A through hole 20 is made in the box-like body 17 of the user interface 16. The box-like body 17 of the user interface 16 is able to be inserted, removably and through the through hole 20, on a support unit 21.

A luminous indicator can be positioned on the box-like body 17 able to indicate with colors and flashing lights the operating state of the apparatus, that is, if the apparatus is ready for collection, if the tube is correctly inserted, whether the collection is in progress or has ended, if there are anomalies, or other. The luminous indicator can also indicate if the user interface 16 has been correctly inserted into the support unit 21.

The support unit 21 is integral with the main body 13 of the apparatus 10 and comprises a clamp or closing gripper 22, which is able to selectively stop the flow of hematic fluid into the transfer tube.

The support unit 21 is preferably made on the front part of the main body 13 of the apparatus 10.

A support rod 23 is also associated to the main body 13, able to accommodate the user interface 16.

The support rod 23 is preferably positioned on the rear part of the main body 13 of the apparatus 10.

The support rod 23 can comprise means 24 for housing instruments 25 which can be used in association with the apparatus 10. The means 24 for housing and supporting instruments 25 can be for example means able to be connected in snap-in manner at the desired height along the support rod.

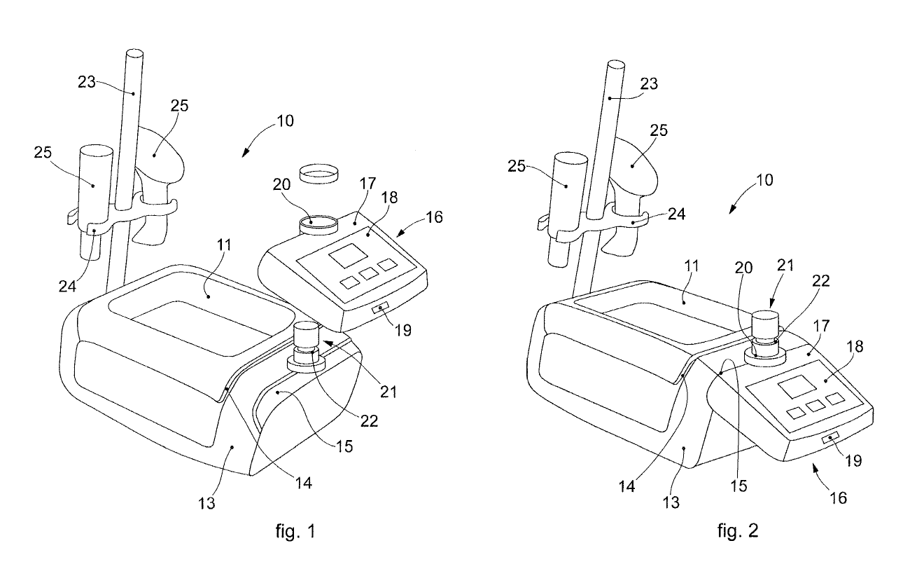

FIG. 2 shows a first positioning mode of the user interface 16 in the present apparatus 10.

In this first configuration of the apparatus 10, the user interface 16 is positioned with at least a part of the box-like body 17 engaged in the seating 15. Moreover, the through hole 20 is inserted into the support unit 21.

This first positioning mode of the user interface 16 is advantageous as it confers compactness on the apparatus 10, as required in mobile use, and can be used, for example, if the operator is sitting in front of the apparatus 10.

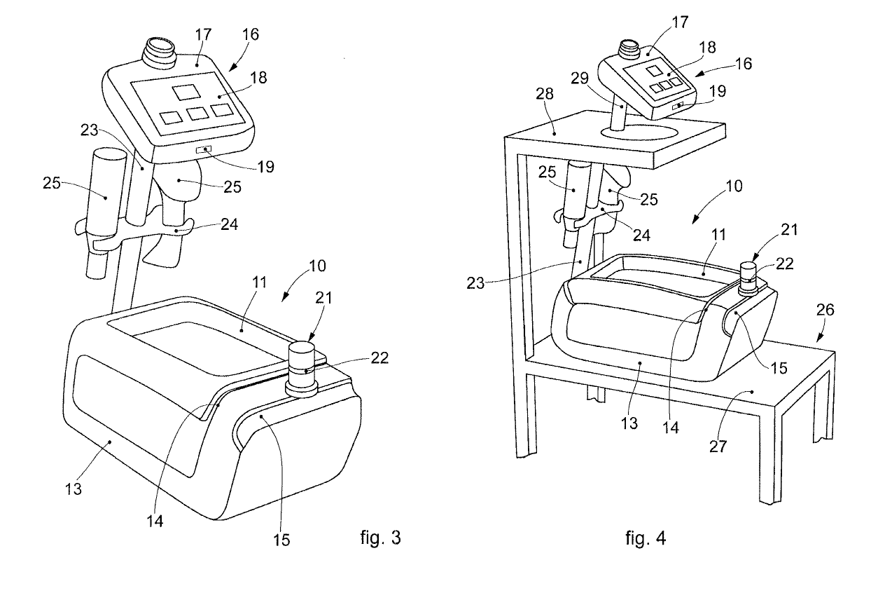

FIG. 3 shows a second configuration of the apparatus 10 in which the user interface 16 is positioned near the top of the support rod 23.

This positioning can be obtained by means of any temporary element or mean to attach the user interface 16 to the support rod 23. For example, it can be provided that an annular positioning element of the user interface 16 is provided on the support rod 23, or a clasp or suchlike, or again a stop element able to stop in position once the user interface 16 has been inserted on the support rod 23, by inserting the corresponding through hole 20.

It is also possible to provide that the user interface 16 on the support rod 23 can be positioned at a variable height on the support rod 23.

The second configuration of the apparatus with the user interface 16 in a raised position with respect to the main body 13 is immediately advantageous if we imagine a standing operator using the apparatus 10 during normal collection and mixing of hematic fluid. In fact, this collection procedure provides to position the main body 13 with the tray 11 in a lower position, so as to obtain a flow of hematic fluid due to the force of gravity. This positioning therefore has undeniable advantages in terms of ergonomics, visibility and efficiency, as it avoids unnecessary bending for the operator in order to access the user interface 16.

Furthermore, advantageously, the user interface 16, as well as in a raised position, is also in a position near to the instruments 25 usable in association with the apparatus 10.

FIG. 4 shows another possible positioning of the user interface 16, which allows another configuration of the apparatus 10.

In this configuration of the apparatus 10, it is provided to use a slider or board 26, comprising a first plane 27 on which the main body 13 rests.

The board 26 comprises a second plane 28 on which the user interface 16 can be positioned.

It is possible to provide that the second plane 28 comprises a support 29 on which the box-like body 17 of the user interface 16 can be inserted, for example in a manner similar to that seen for the support rod 23.

It is possible to provide, for example, that the support 29 is rotatable with respect to the second plane 28.

It is also possible to provide that the board 26 is provided with wheels or suchlike, so that it can be moved.

In this configuration, therefore, the apparatus 10 has a user interface 16 advantageously in a raised position, and it is also provided to use support planes, for example, the first plane 27 and the second plane 28.

The support planes 27 and 28 would also allow to position the main body 13 and the user interface 16 inverted with respect to the one shown, hence with the user interface 16 positioned on the first plane 27 and the main body 13 positioned on the second plane 28.

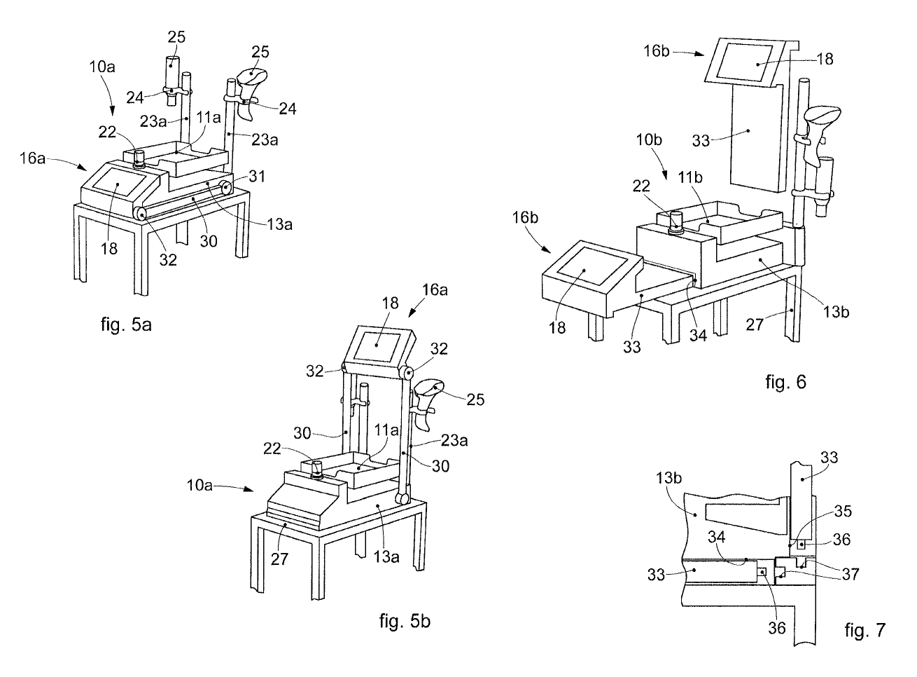

FIG. 5a shows another configuration of the apparatus 10a: in this configuration, the user interface 16a comprises a pair of arms 30 constrained by means of rotation means 31 to the sides of the main body 13a.

As can be seen by comparing FIG. 5a with FIG. 5b, the arms 30 of the user interface 16a can rotate from a substantially horizontal position to a substantially vertical position, or even otherwise inclined, so as to allow to move the user interface to a variable height.

The user interface 16a can be constrained to the arms 30 by means of other rotation means 32, which allow to vary the inclination.

In this case, the apparatus 10a is provided with a pair of support rods 23a able to attach the instruments 25 to be used in association with the apparatus 10a.

FIG. 6 shows another configuration of the apparatus 10b in which the user interface 16b is provided with an appendix 33 configured to be inserted into corresponding seatings 34 and 35 made in the main body 13b of the apparatus 10b.

The seating 34 is substantially horizontal and allows a first positioning of the user interface 16b in a substantially frontal position and similar to the position of FIG. 2 and FIG. 5a.

The seating 35 is substantially vertical and allows a raised positioning of the user interface 16b, similar to the positioning of FIG. 3 or 4 and FIG. 5b.

The appendix 33 can provide, at the free end, positioning pins 36 able to be inserted into corresponding holes 37 made at the bottom of the seatings 34 and 35.

In the embodiment of the apparatus 10b, it is possible to provide, for example, a single support rod 23b on which the instruments 25 are positioned by means of the corresponding housing means 24.

For all the configurations of the apparatus 10, 10a and 10b as described heretofore, the user interface 16 can be connected by means of a suitable electric cable to the rest of the apparatus 10, that is, to the main body 13. The electric cable can be of any type suitable for the purpose, for example, a cable removable from the main body 13, or suchlike.

Alternatively, the connection of the user interface 16 with the main body 13 of the apparatus 10, and hence with the control board integrated therein, can also be obtained by means of wireless connection systems, for example via Bluetooth®, or other radiofrequency systems.

The through hole 20, the rotatable arms 30 and the appendix 33 associated with the user interfaces 16, 16a and 16b are therefore examples of mobile connection means of the user interface with the main body 13, 13a, 13b of the apparatus, so that the user interface 16, 16a, 16b can assume a plurality of different positions, which allow the apparatus to assume different operating configurations.

Naturally, the mobile connection means can be of a different kind to that shown, provided that they guarantee the possibility of using the user interface in multiple positions depending on the different usage requirements, therefore for example a raised position, a lowered position, a position located on a board, or other.

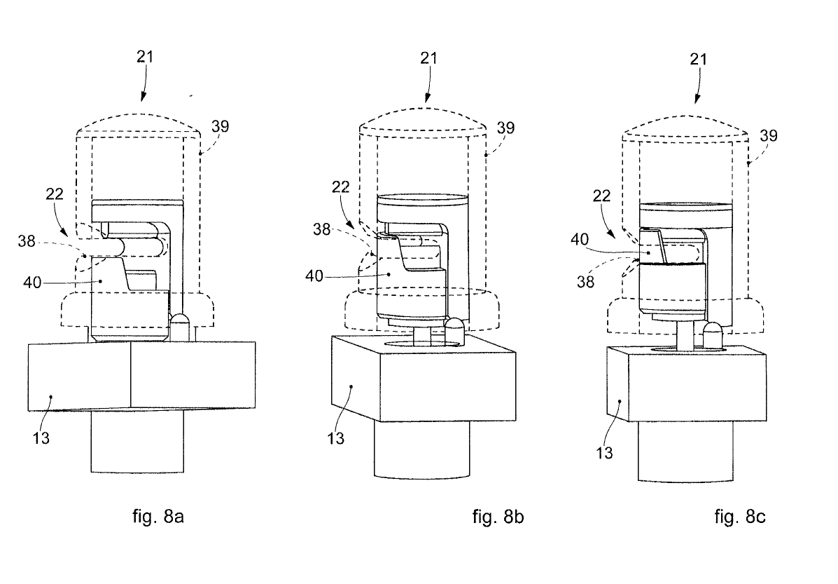

The clamp 22, see FIGS. 8a, 8b and 8c, comprises a casing 39 in which a slit 38 is made, able to accommodate the hematic fluid passage tube.

The clamp 22 is internally provided with at least one mobile division 40 associated with corresponding drive means, for example a stepper motor.

The mobile division 40 can be commanded by the drive means so as to assume at least three positions: a first inactive position, FIG. 8a, a second intermediate holding position, FIG. 3b, and a third position where the hematic fluid passage tube is clamped, FIG. 8c.

In the inactive position shown in FIG. 8a, the mobile division 40 in the lowered position allows the insertion and removal of the hematic fluid passage tube from the slit 38.

In the second intermediate position shown in FIG. 8b, in which the mobile division 40 is in a partly raised position, the hematic fluid passage tube is kept in position and at the same time allows the passage of hematic fluid during the donation.

In the third position shown in FIG. 8c, the mobile division 40 is completely raised and the hematic fluid passage tube is clamped, preventing the passage of hematic fluid.

A detection sensor can also be associated with the clamp 22 comprising the slit 38 and the mobile division 40, and is able to detect the degree of closure or opening of the slit 38, thus able to detect in which position the mobile division 40 is located, and hence in what condition the clamp 22 is operating.

The movement unit 12 of the tray 11, see FIG. 9, is able as we said to allow at least a two-dimensional oscillation of the tray 11, 11a, 11b.

The movement unit 12 comprises a motor member 41 housed on a support frame 42.

The support frame 42 can be housed on the sensor unit 43 which detects the weight force on the tray 11.

The sensor unit 43 is positioned on a support unit 44 and the movement unit 12 is in turn therefore disposed on the sensor unit 43.

The movement unit 12 can include, for example, a rotary disc 45 provided with an eccentric pin 46. The eccentric pin 46 is disposed protruding from the rotary disc 45, parallel to the central axis of the latter.

The rotary disc 45 is connected to the drive shaft of the motor member 41 by which it is made to rotate, and thus can determine the oscillation or tilting of the tray 11.

The drive shaft is rotatably connected to the rotary disc 45, so that the latter is indirectly supported by the support frame 42.

The movement unit 12 is also provided with an oscillation bar 47 connected on one side to the tray 11, and on the other side is slidably connected to the eccentric pin 46.

The oscillation bar 47 is hinged in an intermediate position to the support frame 42 by means of an oscillation or rotation pin 48.

To connect the oscillation bar 47 to the tray 11 a support plate 49 can be provided, attached to the oscillation bar 47 and on which an attachment block 50 is disposed.

The attachment block 50 can advantageously be connected to the tray 11 by means of a magnetic connection device 51.

The magnetic connection device 51 can provide, for example, a first magnetic element associable with the tray 11 and able to cooperate with a second magnetic element associable with the attachment block 50 or to cooperate with the attachment block itself.

The drive of the motor member 41, and hence the rotation of the corresponding drive shaft, determines the rotation of the rotary disc 45 around a drive rotation axis defined by the drive shaft and the eccentric pin 46, which is misaligned with respect to the drive rotation axis, and makes the oscillation bar 47 rotate around the oscillation pin 48.

We must point out here that the axis of rotation defined by the oscillation pin 48 does not coincide with the axis of rotation defined by the drive shaft of the motor member 41, nor with the axis along which the eccentric pin 46 develops.

The oscillation pin 48 in particular is parallel to the axis of rotation defined by the drive shaft, as well as to the axis along which the eccentric pin 46 develops.

In particular, the oscillation bar 47 has a sliding eyelet 52 with an elongated longitudinal shape, inside which the eccentric pin 46 is slidably housed.

In this way the eccentric pin 46 makes the oscillation bar 47 oscillate, causing the alternate oscillation motion of the tray 11 around a single axis defined by the oscillation pin 48.

The movement unit 12 also comprises a sensor member 53 able to detect the position of the tray 11, in particular its inclination.

As mentioned above, the movement unit 12 could also be of another type provided that it guarantees a suitable oscillation to the tray 11.

Around the tray 11 the positioning of an RFID type communication module 54 is also provided, comprising at least one electronic control board 55 to which an antenna 56 is connected.

Advantageously, the antenna 56 is positioned continuously around the tray 11, so as to be present on every side thereof.

The hematic fluid collection sacs will also comprise an RFID type communication module, in particular an electronic label provided with a corresponding antenna and able to communicate with the antenna 56 located near the tray 11.

The antenna 56 is positioned close to the tray 11 by means of a support unit 57 located inside the main body 13, 13a or 13b.

In this way, the data for a particular sac or for every sac of the collection kit, for example data relating to the donor, will automatically be read once the sac has been positioned on the tray 11.

The present apparatus for the collection and mixing of hematic fluid therefore has undeniable advantages from the point of view of ergonomics and from the point of view of operability, since it is provided with various elements to improve its performance, flexibility, safety and in general to simplify its use.

It is clear that modifications and/or additions of parts can be made to the apparatus for the collection and mixing of hematic fluid as described heretofore, without departing from the field and scope of the present invention.

It is also clear that, although the present invention has been described with reference to some specific examples, a person of skill in the art shall certainly be able to achieve many other equivalent forms of apparatus for the collection and mixing of hematic fluid, having the characteristics as set forth in the claims and hence all coming within the field of protection defined thereby.

In the following claims, the sole purpose of the references in brackets is to facilitate reading: they must not be considered as restrictive factors with regard to the field of protection claimed in the specific claims.

1. Apparatus to collect and mix a hematic fluid, comprising a main body, on which at least one tray is positioned, able to receive and support at least a sac connected fluidically to a hematic fluid transfer tube, and at least a user interface to input and display data, wherein said user interface is associated with said main body by means of removable connection means configured to allow said user interface to be disposed in a plurality of different positions with respect to the main body of the apparatus, so that said apparatus can assume different operating configurations.

2. Apparatus as in claim 1, wherein said user interface comprises a box-like body removably associable with a support unit integrated with said main body.

3. Apparatus as in claim 1, wherein said main body comprises one or more support rods configured to house the user interface in a raised position.

4. Apparatus as in claim 1, further comprising a support configured to allow the positioning of the user interface on a board or slider provided with a first plane where the main body can be positioned and a second plane where the user interface can be positioned, or vice versa.

5. Apparatus as in claim 1, wherein said main body is provided with seatings for the insertion of an appendix integrated with the user interface.

6. Apparatus as in claim 1, further comprising a clamp configured to cooperate with the hematic fluid transfer tube and provided with at least a mobile division disposed inside a positioning slit of the tube and associated with corresponding drive means, said mobile division being configured to assume at least a first inactive position, at least an intermediate holding position of the tube and at least a clamping position of the tube.

7. Apparatus as in claim 2, wherein said clamp is positioned on said support unit integrated with the main body of the apparatus.

8. Apparatus as in claim 1, further comprising at least an RFID communication module installable in proximity to the tray and able to read corresponding electronic labels which can be positioned on the hematic fluid collection sacs, in order to carry out an automatic recognition of each sac that transits on the tray.

9. Apparatus as in claim 8, wherein said communication module comprises an RFID antenna that is disposed in proximity to said tray and preferably in proximity to every side thereof.

10. Apparatus as in claim 1, wherein said tray comprises a magnetic device for connection to the main body.

11. Apparatus as in claim 2, wherein said main body comprises one or more support rods configured to house the user interface in a raised position.

12. Apparatus as in claim 2, further comprising a support configured to allow the positioning of the user interface on a board or slider provided with a first plane where the main body can be positioned and a second plane where the user interface can be positioned, or vice versa.

13. Apparatus as in claim 3, further comprising a support configured to allow the positioning of the user interface on a board or slider provided with a first plane where the main body can be positioned and a second plane where the user interface can be positioned, or vice versa.

14. Apparatus as in claim 2, wherein said main body is provided with seatings for the insertion of an appendix integrated with the user interface.

15. Apparatus as in claim 3, wherein said main body is provided with seatings for the insertion of an appendix integrated with the user interface.

16. Apparatus as in claim 4, wherein said main body is provided with seatings for the insertion of an appendix integrated with the user interface.

17. Apparatus as in claim 2, further comprising a clamp configured to cooperate with the hematic fluid transfer tube and provided with at least a mobile division disposed inside a positioning slit of the tube and associated with corresponding drive means, said mobile division being configured to assume at least a first inactive position, at least an intermediate holding position of the tube and at least a clamping position of the tube.

18. Apparatus as in claim 3, further comprising a clamp configured to cooperate with the hematic fluid transfer tube and provided with at least a mobile division disposed inside a positioning slit of the tube and associated with corresponding drive means, said mobile division being configured to assume at least a first inactive position, at least an intermediate holding position of the tube and at least a clamping position of the tube.

19. Apparatus as in claim 4, further comprising a clamp configured to cooperate with the hematic fluid transfer tube and provided with at least a mobile division disposed inside a positioning slit of the tube and associated with corresponding drive means, said mobile division being configured to assume at least a first inactive position, at least an intermediate holding position of the tube and at least a clamping position of the tube.

20. Apparatus as in claim 5, further comprising a clamp configured to cooperate with the hematic fluid transfer tube and provided with at least a mobile division disposed inside a positioning slit of the tube and associated with corresponding drive means, said mobile division being configured to assume at least a first inactive position, at least an intermediate holding position of the tube and at least a clamping position of the tube.
